SEQUENCE LISTING (88 sequences)

SEQ ID NO: 1 - DNA, 20 bp, ARTIFICIAL
  Primer
ctggaacggtgaaggtgaca

SEQ ID NO: 2 - DNA, 23 bp, ARTIFICIAL
  Primer
aagggacttcctgtaacaatgca

SEQ ID NO: 3 - DNA, 20 bp, ARTIFICIAL
  Primer
gcaaggctgacgataaggag

SEQ ID NO: 4 - DNA, 20 bp, ARTIFICIAL
  Primer
tggctttacaccaacgaaaa

SEQ ID NO: 5 - DNA, 20 bp, ARTIFICIAL
  Primer
ctttgggctgctcgctatga

SEQ ID NO: 6 - DNA, 20 bp, ARTIFICIAL
  Primer
tggcttggaaagttcgggtc

SEQ ID NO: 7 - DNA, 21 bp, ARTIFICIAL
  Primer
gctgcctttcatttagcactc

SEQ ID NO: 8 - DNA, 20 bp, ARTIFICIAL
  Primer
ctggtgggttgaggagagaa

SEQ ID NO: 9 - DNA, 20 bp, ARTIFICIAL
  Primer
gtcacactggctctctgctg

SEQ ID NO: 10 - DNA, 20 bp, ARTIFICIAL
  Primer
tgatgctttctctgggcttt

SEQ ID NO: 11 - DNA, 20 bp, ARTIFICIAL
  Primer
aagactcggaccaaggacaa

SEQ ID NO: 12 - DNA, 20 bp, ARTIFICIAL
  Primer
tgttgctgctgctgtttctt

SEQ ID NO: 13 - DNA, 18 bp, ARTIFICIAL
  Primer
acctgtgcgagtggatgc

SEQ ID NO: 14 - DNA, 19 bp, ARTIFICIAL
  Primer
tcctttgctctgcggttct

SEQ ID NO: 15 - DNA, 20 bp, ARTIFICIAL
  Primer
ggggaaaaccaggacaaaag

SEQ ID NO: 16 - DNA, 19 bp, ARTIFICIAL
  Primer
gcaccgagcctccactatt

SEQ ID NO: 17 - DNA, 20 bp, ARTIFICIAL
  Primer
cttctcagggggtcatcttg

SEQ ID NO: 18 - DNA, 20 bp, ARTIFICIAL
  Primer
tcccaaagcaaaggttgttc

SEQ ID NO: 19 - DNA, 20 bp, ARTIFICIAL
  Primer
tcccagctcctcatgtatcc

SEQ ID NO: 20 - DNA, 20 bp, ARTIFICIAL
  Primer
acttgatgccctggctgtag

SEQ ID NO: 21 - DNA, 19 bp, ARTIFICIAL
  Primer
caccgcatctggagaacca

SEQ ID NO: 22 - DNA, 21 bp, ARTIFICIAL
  Primer
gcccatttcctcggtgtagtt

SEQ ID NO: 23 - DNA, 18 bp, ARTIFICIAL
  Primer
aaacgggggcttcttcct

SEQ ID NO: 24 - DNA, 21 bp, ARTIFICIAL
  Primer
cggttagcacacactcctttg

SEQ ID NO: 25 - DNA, 20 bp, ARTIFICIAL
  Primer
gactacctgctgggcatcaa

SEQ ID NO: 26 - DNA, 20 bp, ARTIFICIAL
  Primer
tgcactcatcggtgaagaag

SEQ ID NO: 27 - DNA, 20 bp, ARTIFICIAL
  Primer
cctgagcgacacacaagaag

SEQ ID NO: 28 - DNA, 20 bp, ARTIFICIAL
  Primer
ttcactttccacccctttga

SEQ ID NO: 29 - DNA, 20 bp, ARTIFICIAL
  Primer
ggacacctttggaagcagag

SEQ ID NO: 30 - DNA, 20 bp, ARTIFICIAL
  Primer
ccagcacaatctccgtgaag

SEQ ID NO: 31 - DNA, 21 bp, ARTIFICIAL
  Primer
atgaacaagaaggggaaactc

SEQ ID NO: 32 - DNA, 21 bp, ARTIFICIAL
  Primer
ttggaagaaagtgagcagagg

SEQ ID NO: 33 - DNA, 18 bp, ARTIFICIAL
  Primer
gccaggacccgaacagag

SEQ ID NO: 34 - DNA, 19 bp, ARTIFICIAL
  Primer
cccagaagaggaggcactt

SEQ ID NO: 35 - DNA, 20 bp, ARTIFICIAL
  Primer
tcctgaggagcagatgacct

SEQ ID NO: 36 - DNA, 20 bp, ARTIFICIAL
  Primer
ccgaaggaccagacatcact

SEQ ID NO: 37 - DNA, 18 bp, ARTIFICIAL
  Primer
gcctcctcggagtccttg

SEQ ID NO: 38 - DNA, 20 bp, ARTIFICIAL
  Primer
atccttgacccagacagtgg

SEQ ID NO: 39 - DNA, 19 bp, ARTIFICIAL
  Primer
aggcagttggtgggaagtc

SEQ ID NO: 40 - DNA, 20 bp, ARTIFICIAL
  Primer
ggctactgtcagctcctgct

SEQ ID NO: 41 - DNA, 20 bp, ARTIFICIAL
  Primer
aggaggaaaacgggaaagaa

SEQ ID NO: 42 - DNA, 20 bp, ARTIFICIAL
  Primer
caacaacagcaatggaggag

SEQ ID NO: 43 - DNA, 21 bp, ARTIFICIAL
  Primer
gaggagaaagtggaggtctgg

SEQ ID NO: 44 - DNA, 21 bp, ARTIFICIAL
  Primer
gcaagaaagtagcatcgtctg

SEQ ID NO: 45 - DNA, 20 bp, ARTIFICIAL
  Primer
gcttcagaacccatccatgt

SEQ ID NO: 46 - DNA, 20 bp, ARTIFICIAL
  Primer
ttcccctcacgaagaagttg

SEQ ID NO: 47 - DNA, 19 bp, ARTIFICIAL
  Primer
ctgcctaagatgcccgact

SEQ ID NO: 48 - DNA, 20 bp, ARTIFICIAL
  Primer
ctttttgctgcgtttccatt

SEQ ID NO: 49 - DNA, 21 bp, ARTIFICIAL
  Primer
catggccaagattgacaacct

SEQ ID NO: 50 - DNA, 22 bp, ARTIFICIAL
  Primer
ttcccatatgttcctgcatcag

SEQ ID NO: 51 - DNA, 20 bp, ARTIFICIAL
  Primer
cggaccccattctctcttct

SEQ ID NO: 52 - DNA, 20 bp, ARTIFICIAL
  Primer
acttcgtcttccgaggctct

SEQ ID NO: 53 - DNA, 20 bp, ARTIFICIAL
  Primer
accaggacaccatgaggaac

SEQ ID NO: 54 - DNA, 20 bp, ARTIFICIAL
  Primer
cgccgacaggtacttctgtt

SEQ ID NO: 55 - DNA, 20 bp, ARTIFICIAL
  Primer
attcgttggggatgacagag

SEQ ID NO: 56 - DNA, 20 bp, ARTIFICIAL
  Primer
cgagtcctgcttcttcttgg

SEQ ID NO: 57 - DNA, 20 bp, ARTIFICIAL
  Primer
cgaaagagaaagcgaaccag

SEQ ID NO: 58 - DNA, 20 bp, ARTIFICIAL
  Primer
aaccacactcggaccacatc

SEQ ID NO: 59 - DNA, 18 bp, ARTIFICIAL
  Primer
cggaggatgtggaagtgg

SEQ ID NO: 60 - DNA, 21 bp, ARTIFICIAL
  Primer
tttggatggacgcttattttc

SEQ ID NO: 61 - DNA, 20 bp, ARTIFICIAL
  Primer
ggggcacagacttcctttta

SEQ ID NO: 62 - DNA, 19 bp, ARTIFICIAL
  Primer
ctcccgttcacgaacactc

SEQ ID NO: 63 - DNA, 19 bp, ARTIFICIAL
  Primer
cccatggatgaagtctacc

SEQ ID NO: 64 - DNA, 19 bp, ARTIFICIAL
  Primer
gtcctcctcctttttccac

SEQ ID NO: 65 - DNA, 20 bp, ARTIFICIAL
  Primer
ggctaccctgagactgacca

SEQ ID NO: 66 - DNA, 21 bp, ARTIFICIAL
  Primer
cacagggcatcttttccataa

SEQ ID NO: 67 - DNA, 20 bp, ARTIFICIAL
  Primer
gccatcggctacatcaactt

SEQ ID NO: 68 - DNA, 19 bp, ARTIFICIAL
  Primer
ggagggaggccataatcag

SEQ ID NO: 69 - DNA, 19 bp, ARTIFICIAL
  Primer
gcgaggatggcaagaaaag

SEQ ID NO: 70 - DNA, 20 bp, ARTIFICIAL
  Primer
agatttgacgaaggcgaaga

SEQ ID NO: 71 - DNA, 21 bp, ARTIFICIAL
  Primer
caagcagtttatccccaatgt

SEQ ID NO: 72 - DNA, 19 bp, ARTIFICIAL
  Primer
gtcacccgcagtttcactc

SEQ ID NO: 73 - DNA, 20 bp, ARTIFICIAL
  Primer
tacctcttcctcccactcca

SEQ ID NO: 74 - DNA, 20 bp, ARTIFICIAL
  Primer
cccatttccctcgtttttct

SEQ ID NO: 75 - DNA, 20 bp, ARTIFICIAL
  Primer
gaggaagtcggtgaagaacg

SEQ ID NO: 76 - DNA, 20 bp, ARTIFICIAL
  Primer
ccaacatcgagaccttcgat

SEQ ID NO: 77 - DNA, 18 bp, ARTIFICIAL
  Primer
aagggcgagtcccgtatc

SEQ ID NO: 78 - DNA, 20 bp, ARTIFICIAL
  Primer
ttgtagttggggtggtcctg

SEQ ID NO: 79 - DNA, 20 bp, ARTIFICIAL
  Primer
cctctgtcccctctccctac

SEQ ID NO: 80 - DNA, 20 bp, ARTIFICIAL
  Primer
ctccagaaccatctccgtgt

SEQ ID NO: 81 - DNA, 20 bp, ARTIFICIAL
  Primer
ttgcacatcgcagaaagaag

SEQ ID NO: 82 - DNA, 20 bp, ARTIFICIAL
  Primer
gtgtttcggatggctctgat

SEQ ID NO: 83 - DNA, 20 bp, ARTIFICIAL
  Primer
agaagagcctgtcgctgaaa

SEQ ID NO: 84 - DNA, 20 bp, ARTIFICIAL
  Primer
ttggaccagaaggagcagtc

SEQ ID NO: 85 - DNA, 20 bp, ARTIFICIAL
  Primer
ctctggctttgaccctgaac

SEQ ID NO: 86 - DNA, 20 bp, ARTIFICIAL
  Primer
tcctctcttttgcctggatg

SEQ ID NO: 87 - DNA, 20 bp, ARTIFICIAL
  Primer
ctgagaggaggaaggtgctg

SEQ ID NO: 88 - DNA, 20 bp, ARTIFICIAL
  Primer
aggaggccaggctctatttc

(19) United States
(12) Patent Application Publication
(10) Pub. No.: US 2012/0135519 A1
(43) Pub. Date: May 31, 2012

(54) INDUCED DERIVATION OF SPECIFIC ENDODERM FROM hPS CELL-DERIVED DEFINITIVE ENDODERM

(75) Inventors: Jacqueline Ameri, Malmo (SE); Henrik Semb, Bjarred (SE)

(73) Assignees: Cellartis AB, Gothenburg (SE); Novo Nordisk A/S, Bagsvaerd (DK)

(21) Appl. No.: 13/322,175

(22) PCT Filed: May 28, 2010
(86) PCT No.: PCT/EP10/57465

§ 371 Date: Feb. 6, 2012

Related U.S. Application Data
(60) Provisional application No. 61/182,142, filed on May 29, 2009.

(51) Int. Cl. C12N 5/071

(52) U.S. Cl. 435/366; 435/375

(57) ABSTRACT

The present invention relates to a method to control differentiation of human pluripotent stem cells, including human balstocyst derived stem (hBS) cells and to obtain specific endoderm cells. In particular, present invention relates to the use of FGF2 as the key factor in a specific concentration to control differentiation of definitive endoderm cells derived from hPS cells to specific endoderm cells. The invention also provides methods of obtaining endoderm cells comprising the use of FGFR and activation of the MAPK signalling pathway.

### TECHNICAL FIELD

The present invention relates to a method to control differentiation of human pluripotent stem cells, including human balstocyst derived stem (hBS) cells and to obtain specific endoderm cells.

### BACKGROUND OF THE INVENTION

The foregut derivatives pancreas, lung, thyroid, liver, esophagus, and stomach originate from definitive endoderm, one of the three germ layers that form during gastrulation Specific transcription factors are expressed in a specific manner along the anterior and posterior axis (A-P axis) of the definitive endoderm, which eventually forms the primitive gut tube. Forkhead box A1 (FOXA1) and FOXA2 are both expressed in the entire gut tube and are thus important for development of all gastrointestinal tract derived organs (Ang et al., 1993). In the anterior portion of foregut endoderm, regions that are destined to become lung and thyroid express NK2 homeobox 1 (NKX2.1), whereas liver develops from a region expressing hematopoietically expressed homeobox (HHEX1). Pancreas and duodenum originate from the posterior portion of foregut endoderm expressing pancreas duodenum homeobox 1 (PDX-1). The posterior portion of gut endoderm develops into mid- and hindgut that become the small and large intestine, expressing caudal type homeobox 1 (CDX1) and CDX2.

The Fibroblast growth factor (FGF) family is controlling many aspects of development, such as cell migration, proliferation, and differentiation. There are at least four different tyrosine kinase receptors (FGFR1-FGFR4) that bind different FGF ligands with varying affinities. In addition, alternative splicing of FGFR1-FGFR3 generates ‘IIIb’ and ‘IIIc’ isoforms, which have separate expression patterns and ligand specificities FGF signaling has been implicated in patterning of the gut tube along the A-P axis and during pancreatic differentiation.

Prior studies involving mouse and chick embryo explants have established that FGF1 and FGF2 are secreted by the cardiac mesoderm and that it can be replaced by exogenous addition of these factors. During early embryogenesis, the ventral endoderm lies adjacent to the cardiac mesoderm, while the dorsal endoderm is in contact with the notochord. Cardiac mesoderm is required for liver and lung development. Specifically, FGF2 patterns the multipotent ventral foregut endoderm in a concentration-dependent manner into liver and lung, while the absence of cardiac mesoderm and FGFs promotes a pancreatic fate. Although, the presence of FGF2 is not absolutely required for ventral pancreas development, an inductive role during dorsal pancreas formation has been demonstrated. Dorsal endoderm is initially in contact with the notochord that secretes Activin βB and FGF2, resulting in inhibition of Shh expression, which is required for Pdx1 expression and dorsal pancreas development. In addition, low levels of FGF2 induce Pdx1 expression in cultured chick dorsal endoderm. Furthermore, FGF2 has also been suggested to have an inductive effect on the proliferation of pancreatic epithelial cells in the developing pancreas and is expressed together with other FGFs in adult mouse beta cells.

Increased prevalence of type I diabetes and lack of cadaveric donor islets has created great interest in developing protocols for directing differentiation of human blastocyst stem cells (hBS cells) into insulin producing beta cells. To better understand the molecular mechanisms of cell fate specification of ES cells towards pancreatic endoderm and insulin expressing cells, refined culture conditions are needed. While a number of differentiation protocols have been published reporting in vitro derivation of insulin producing cells from hPS cells, none of these describe the specific role of the individual growth factors employed in the differentiation process or discuss underlying molecular mechanisms. In addition, it is not clear if these insulin-expressing cells represent bona fide beta cells. In our efforts to understand the conversion of hPS cell-derived definitive endoderm into PDX1 positive beta cell progenitors, we examined the role of FGF2.

Our results indicate that in the absence of exogenous FGF2, definitive endoderm differentiate into foregut and midgut endoderm characterized by hepatocytes and intestinal-like cells. Importantly, exogenously added FGF2 patterns hPS cell derived definitive endoderm in a dose-dependent manner. Specifically, hepatic, pancreatic, intestinal, and anterior foregut progenitors are generated in response to distinct FGF2 concentrations. Moreover, the stepwise addition of growth factors allowed us to further dissect the molecular program that regulates pancreas specification, showing that induction of pancreatic progenitors/PDX1 expression relies on the FGF2-mediated activation of the MAPK signalling pathway. This is the first time that FGF2 alone has been implicated in the differentiation of hPS cell derived pancreatic endoderm; prior to this, methods for deriving pancreatic endoderm relied on culturing cells in the presence of combinations of growth factors, such as FGF members with retinoates (see WO 07/127927) or in the presence of these growth factors with additional media supplements such as B27 (WO 09/012428). The data shown here will therefore be instrumental for developing novel and reproducible protocols for inducing hPS cells towards the anterior and posterior endoderm derivatives lung, esophagus, stomach, liver, pancreas, and intestine.

As mentioned above, current knowledge regarding differentiation of hPS cell into pancreatic mainly comprise studies on chicken, mice and to a limited extent human cells. Although hPS cell differentiation protocols have been reported, it is not clear if these insulin-expressing cells represent bona fide beta cells due to their low insulin content and lack of physiological glucose-mediated insulin release. The fact that the protocols vary in growth factor composition, concentration and timing of addition, suggest that there is a need to precisely define the specific role and mode of action of individual growth factors in this differentiation process in order to provide a method by which cell-differentiation is controlled.

### DESCRIPTION OF THE INVENTION

Present invention relates to the use of FGF2 as the key factor in a specific concentration to control differentiation of definitive endoderm cells derived from hPS cells to specific endoderm cells.

The invention also provides methods of obtaining endoderm cells comprising the use of FGFR and activation of the MAPK signalling pathway.

As schematically depicted in FIG. 1A, the differentiation procedure may comprise one or more steps, such as two steps which include a first step, directing differentiation towards definitive endoderm, while the second step directs the further differentiation towards specific endoderm.

The first step, which facilitates differentiation into definitive endoderm may comprise different growth media compositions that are changed during the first step, as schematically depicted in FIG. 1A and exemplified in Example 2.

Present invention relates preferentially to the second step, starting from definitive endoderm cells. To direct the differentiation into specific endoderm cells, a number of conditions are necessary to ensure growth and viability. Furthermore key components as growth factors are necessary to control differentiation.

In present invention, differentiation of definitive endoderm cells is directed to certain types of specific endoderm cells by subjecting the definitive endoderm cells to different concentrations of the fibroblast growth factor, FGF2. Low concentrations of FGF2 leads to hepatic endodermal cells, medium concentrations of FGF2 leads to pancreatic endodermal cells, whereas relative high concentrations of FGF2 leads to intestinal and/or lung endodermal cells or mixtures thereof. The concentration of FGF2 is the concentration in the culture medium and is in the range of from 0.1 to 500 ng/ml.

To guide differentiation towards a hepatic cell fate FGF2 may be added in the culture media in ranges from 0.1-16 ng/ml, or 0.1-10 ng/ml. This results in the generation of hepatic endodermal cells that express AFP and one or more markers selected from FOXA2, Albumin (ALB), HNF4A, HNF6 (ONECUT1), Prox1, CK17, CK19, Hex, FABp1, AAT, Cyp7A1, Cyp3A4, Cyp3A7 and Cyp2B6 are expressed in hepatic endodermal cells. In general the hepatic endodermal cells express the following markers: AFP, ALB, HNF6 and HNF4A and/or AFP, HNF4A, Prox1. In one aspect of the invention, the concentration of FGF2 is in a range from 4 ng/ml to 6 ng/ml, such as 5 ng/ml, and the specific endoderm cells are hepatic endoderm cells

Normally, the hepatic endodermal cells express AFP and at least 4, at least 5, at least 6 such as at least 7, at least 8, at least 8, at least 9, at least 10, at least 11, at least 12 or all of the above-mentioned markers are expressed by the hepatic endodermal cells obtained.

As disclosed herein the hepatic endodermal cells obtained by subjecting definitive endodermal cells to a low concentration of FGF2 (0.1-16 ng/ml) express AFP, ALB, ONECUT1, HNF4A.

Based on morphologic studies hepatocyte-like cells were clearly observed in cultures treated with only Activin A or low FGF2 concentrations such as 4 ng/ml, whereas these cells were not seen at higher concentrations of FGF2, such as 16-256 ng/ml. Additionally, with increasing FGF2 concentrations, colonies got denser and thick clusters appeared.

As illustrated in FIG. 1. B) the amount of albumin (ALB) expressing cells decreases with increasing FGF2 concentrations. Furthermore, antibody staining (not shown) revealed consistent coexpression of ALB and AFP. Hepatocyte associated markers ALB, HNF4A and ONECUT are downregulated with increasing FGF-concentrations, compared to reference samples treated with only Activin A. Thus one aspect of the invention relates to the use of FGF2 for controlling (i.e. promoting or inhibiting) the differentiation of hPS cells towards a hepatic cell fate.

To guide differentiation of the DE-cells towards pancreatic endoderm, FGF2, when added to the culture media in ranges from 16-150 ng/ml, such as 64 ng/ml, stimulates the formation of pancreatic endodermal cells. The pancreatic endodermal cells obtained express PDX-1 and one or more of the following markers NGN3, CPA1, SOX9, HNF6, HNF1b, E-cadherin, MNX1, PTF1A and NKX6-1. In general the pancreatic endodermal cells express PDX1 and at least 2, at least 3, at least 4, at least 5, at least 6, at least 7, at least 8 or all of the above markers.

As seen from the examples herein, the pancreatic endodermal cells obtained express PDX1 and NKX6-1, and/or PDX1, SOX9, ONECUT1, and FOXA2.

Furthermore, the pancreatic endoderm cells express at least one pancreatic hormone selected from the group consisting of insulin, glucagon, somatostatin, pancreatic polypeptide, and ghrelin.

To guide the differentiation of the definitive endoderm cells towards intestinal and/or lung endoderm, FGF2 is added to the culture media in ranges from 150-500 ng/ml.

Intestinal endodermal cells obtained express CDX2 and one or more of the following markers CDX1, FOXA2, PITX2, FABp2, TCF4, Villin and MNX1. In general, the intestinal endodermal cells obtained express CDX1 and at least 2, at least 3, at least 4, at least 5, at least 6 or all of the above-mentioned markers. From the examples herein it is shown that the intestinal endodermal cells obtained express CDX1, CDX2 and MNX1.

Lung endodermal cells obtained that express one or more of the following markers NKX2-1, SHH, PTCH1, FGF10, and SPRY2. In general the lung endodermal cells obtained express at least 2, at least 3 or all of the above-mentioned markers.

Anterior foregut endodermal cells obtained expressing SOX2.

When FGF2 is used in a concentration of from 150-500 ng/ml it is contemplated that intestinal endodermal cells predominantly are obtained using a concentration in the lower end of the range and lung endodermal cells predominantly are obtained using a concentration in the higher end of the range. Mixtures of intestinal and lung endodermal cells may also be obtained.

Starting material for obtaining specific endodermal cells is definitive endodermal cells. Definitive endodermal cells can be obtained by subjecting hPS cells to a suitable protocol (see e.g. FIG. 1A first two columns) or Example 2 or definitive endoderm may be obtained by other types of pluripotent cell lines such as iPS-cells or cells showing the potential to differentiate into definitive endoderm.

The definitive endodermal cells are characterized by expression of the following markers SOX17, FOXA2, CXCR4 and down regulation of the marker SOX7.

More specifically, the definitive endodermal cells co-express SOX17 and CXCR4 at a protein level and; show gene expression of cereberus, Foxa2, GSC, HHEX. Oct-4 is down regulated at day 3 in Activin A treated samples (cf. example 3).

The definitive endodermal cells are subjected to culturing in a suitable medium in the presence of a selected concentration of FGF2 as described above in order to direct the development of the definitive endodermal cells into specific endodermal cells, cf. above. More details are given in the examples herein. In short, differentiation of definitive endodermal cells is induced by culturing the cells in a suitable medium (e.g. KO-DMEM medium) containing FGF for up to 20 days, such as 8-12 days, the medium optionally containing an antibiotic (e.g. Penicillin-streptomycin e.g. in a concentration of 1%), one or more nutrients or other substances normally present in culture medium (e.g. 1% of Glutamax, 1% non-essential amino acids, 0.1 mM beta-Mercaptoethanol) and knockout serum replacement (e.g. 10-15% such as 12%). The medium is kept fresh and with even concentration levels over time.

A significant aspect of the invention, which allows a precise and simple guidance of stem cell differentiation, is the finding that FGF2 alone is sufficient for induction of pancreas specific genes and.

As illustrated in FIG. 2. PDX1, SOX9 and NGN3 are up-regulated in all of the FGF2 treated samples except for PDX1 when treated with only 4 ng/ml FGF2, which remain unchanged in comparison with the control sample. When treated with 64 ng/ml FGF2, NGN3, was up regulated but to a lower degree than at 32 ng/ml FGF2 and 256 ng/ml FGF2 possibly indicating a negative correlation between the expression of PDX1/NKX6-1 and NGN3 or possibly indicating that the PDX1/NKX6.1+ cells are more abundantly present at 64 ng/ml FGF2 than cells expressing higher levels of NGN3. Both NKX6-1 and PDX1 show peak expression in samples in which FGF2 is added in a concentration around 64 ng/ml. These observations are further supported by immuno fluorescence stainings of PDX1+ colonies at 64 ng/ml FGF2, showing corresponding patterns. Furthermore, it is apparent that all PDX1+ cells are SOX9, ONECUT1 and FOX2A positive, while the majority of the PDX1+ cells are negative for the intestine marker CDX2 and the proliferation marker PH-3. Some cells expressing both PDX1 and NKX6-1 may be found within the PDX1 positive colonies.

To allow efficient differentiation of the DE cells to specialized endoderm cells, different concentrations of FGF2 is added to the DE cells. To reveal transcriptional changes in response to FGF2 concentrations, the expression pattern is monitored by RNA analysis. The result, as depicted in FIG. 3 clearly shows that FGF2 directs differentiation since the lung associated markers including NKX2-1, SHH, PTCH1, SPRY2 and FGF10 and the small intestine marker CDX2 and MNX1 all show a distinct upregulation and peak expressions at 256 ng/ml FGF2. Contrary to CDX2, the small intestine marker CDX1 remains unaffected of FGF2 level, in the range tested.

Supporting immunofluorescence studies of the PDX1 positive population at 256 ng/ml FGF2 further reveal that all PDX1 positive cells are SOX9 and ONECUT1 positive while only few PDX1+ cells were CDX2 positive. None of the PDX1+ cells coexpressed NKX6-1 or SOX2. In addition, SOX2+ cells were CDX2 negative.

Furthermore, immunofluorescence double stainings reveal that almost all CDX2 positive cells coexpress FOXA2, when grown at 256 ng/ml FGF2 whereas only a few CDX2+ cells express MK167.

As depicted in FIG. 4A, FGF2 affects the transcription of FGFR (FGF-receptor) genes in a dose dependent manner. As apparent from FIG. 4A, FGFR1 and -3 are upregulated in response to increasing FGF2 concentration while FGFR2 and -4 show the opposite mechanism, with decreasing transcription levels as a consequence of increasing FGF2 levels.

The present invention also provides i) a method for the preparation of hepatic endodermal cells, the method comprising incubating definitive endodermal cells in a culture medium containing from 0.1 to 16 ng/ml FGF2 for about 6 to 20 days such as 6 to 8 days or 9 to 12 days, ii) hepatic endodermal cells obtainable by such a method and iii) hepatic endodermal cells obtained by such a method and having the characteristics as defined herein.

Moreover, the present invention also provides i) a method for the preparation of pancreatic endodermal cells, the method comprising incubating definitive endodermal cells in a culture medium containing from 16 to 150 ng/ml FGF2 for about 2 to 20 days such as 6 to 8 days, ii) pancreatic endodermal cells obtainable by such a method and iii) pancreatic endodermal cells obtained by such a method and having the characteristics as defined herein.

Furthermore, the present invention also provides i) a method for the preparation of intestinal and/or lung endodermal cells, the method comprising incubating definitive endodermal cells in a culture medium containing from 150 to 500 ng/ml FGF2 for about 6 to 20 days such as 6 to 8 days, ii) intestinal and/or lung endodermal cells obtainable by such a method and iii) intestinal and/or lung endodermal cells obtained by such a method and having the characteristics as defined herein.

It is hypothesized that the method for the preparation of hepatic, pancreatic or intestinal endodermal cells comprising inducing FGFR, notably FGFR is FGFR1,FGFR2, FGFR3 and/or FGFR4.

FGFR is induced by addition of a FGF to a culture of definitive endoderm cells. A suitable FGF may be selected from FGF2 alone or in combination with a second FGF chosen from the following: FGF4, FGF7, and FGF10, and any combination thereof. Studies performed by the applicant have shown that neither FGF4, FGF7 nor FGF10, when used alone instead of FGF2, is capable of inducing differentiation of hPS-derived definitive endoderm towards PDX-1 positive pancreatic endoderm. As described in FIGS. 4B and C it is envisaged that MAPK signalling pathway is activated by FGFR-induction.

### BRIEF DESCRIPTION OF THE DRAWINGS

FIG. 1. A) A schematic representation of the two-step differentiation procedure towards specified endoderm. The differentiation protocol is divided into two steps: the first step directs differentiation towards definitive endoderm, while the second step directs differentiation towards specified endoderm. B) Hepatocyte associated markers ALB, HNF4A, and ONECUT1 were all downregulated with increasing FGF2 concentrations (ng/ml) in comparison to the control sample treated only with Activin A. As HHEX is also expressed in the anterior foregut endoderm it was not downregulated in the same extent as the other hepatic markers at the highest FGF2 concentration 256 ng/ml. Samples were taken for real-time PCR analysis at day eleven. The data is shown as mean expression +/−SEM (n=4). The graphs represent the fold increase in comparison to that detected in the control samples at day eleven. The control sample was arbitrarily set to a value of one.

FIG. 2. A) FGF2 is sufficient for the induction of pancreas specific genes. PDX1, SOX9 and NGN3 were upregulated in all of the FGF2 treated samples except for PDX1 when treated with only 4 ng/ml FGF2, which remained unchanged in comparison with the control sample. When treated with 64 ng/ml, NGN3, was up regulated but to a lower degree than at 32 ng/ml and 256 ng/ml possibly indicating a negative correlation between the expression of PDX1/NKX6-1 and NGN3 or possibly indicating that the PDX1/NKX6.1+ cells are more abundantly present at 64 ng/ml FGF2 than cells expressing higher levels of NGN3. NKX6-1 was only upregulated at and 64 ng/ml. Both PDX1 and NKX6-1 had peak expression at 64 ng/ml. FOXA2 and CPA1 were detected in all of the samples and remained unchanged. Samples were taken for real-time PCR analysis at day eleven. The data is shown as mean expression +/−SEM (n=4). The graphs represent the fold increase in comparison to that detected in the control samples at day eleven. The control sample was arbitrarily set to a value of one.

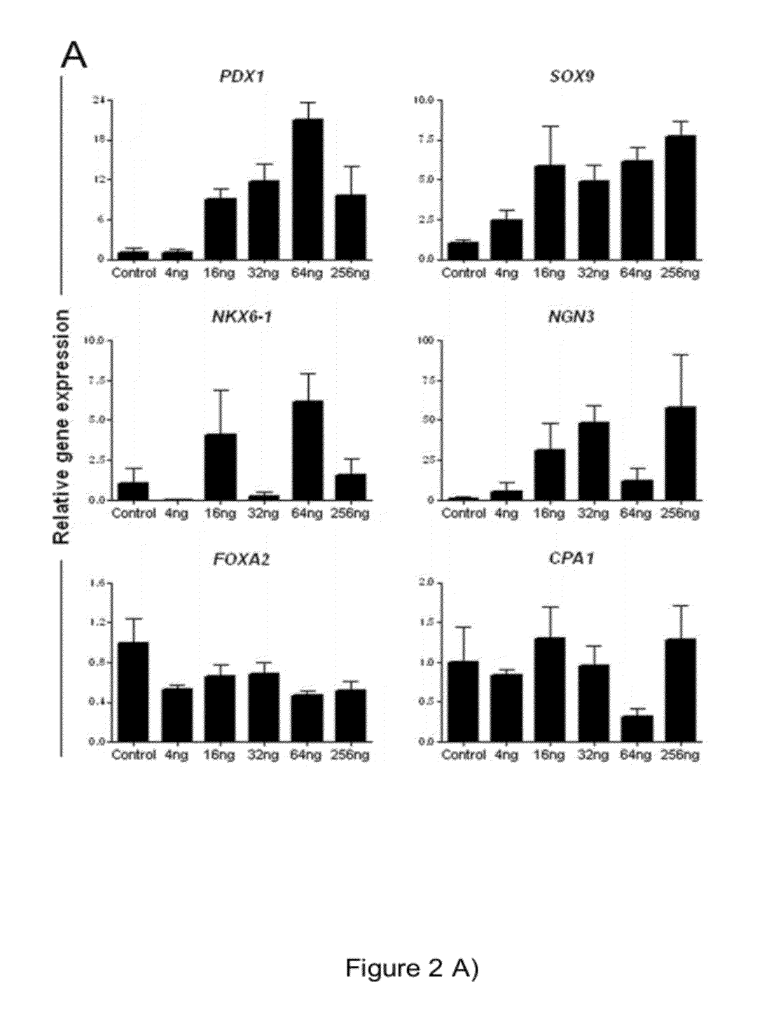

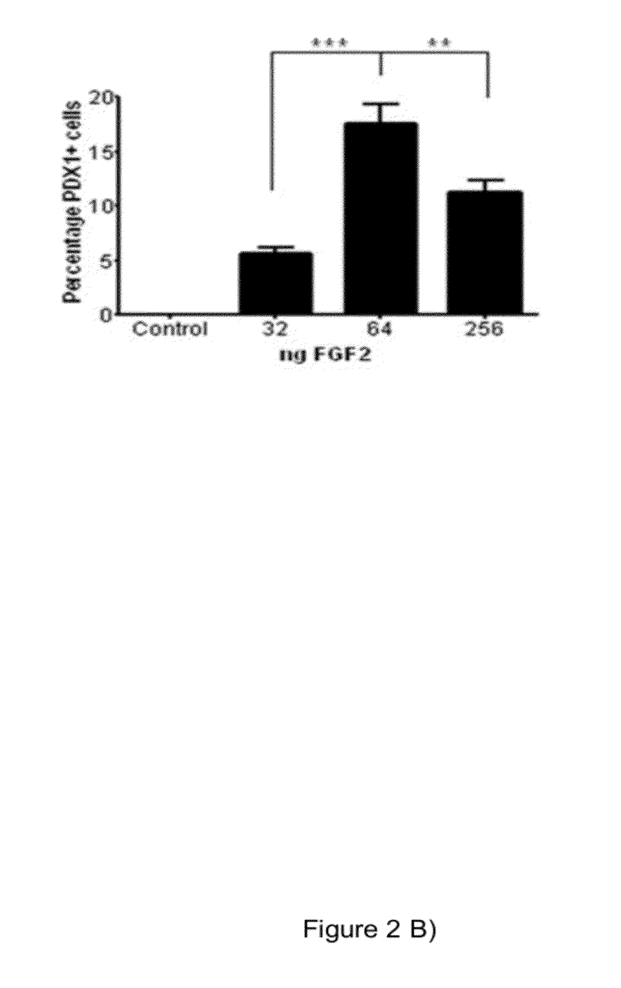

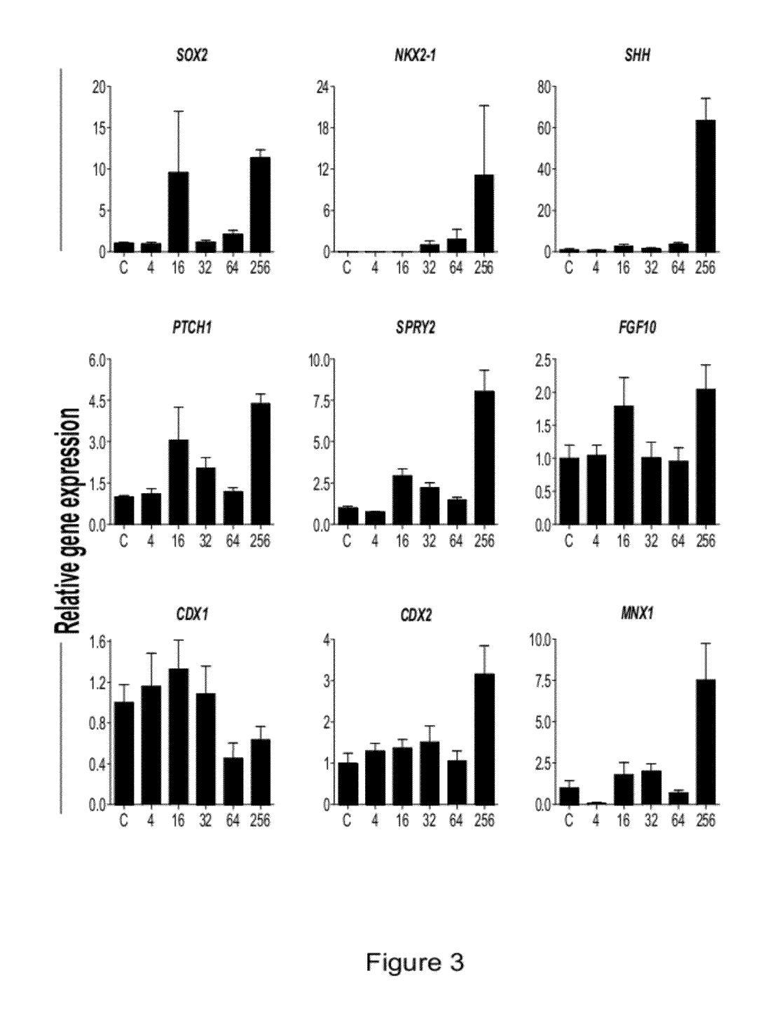

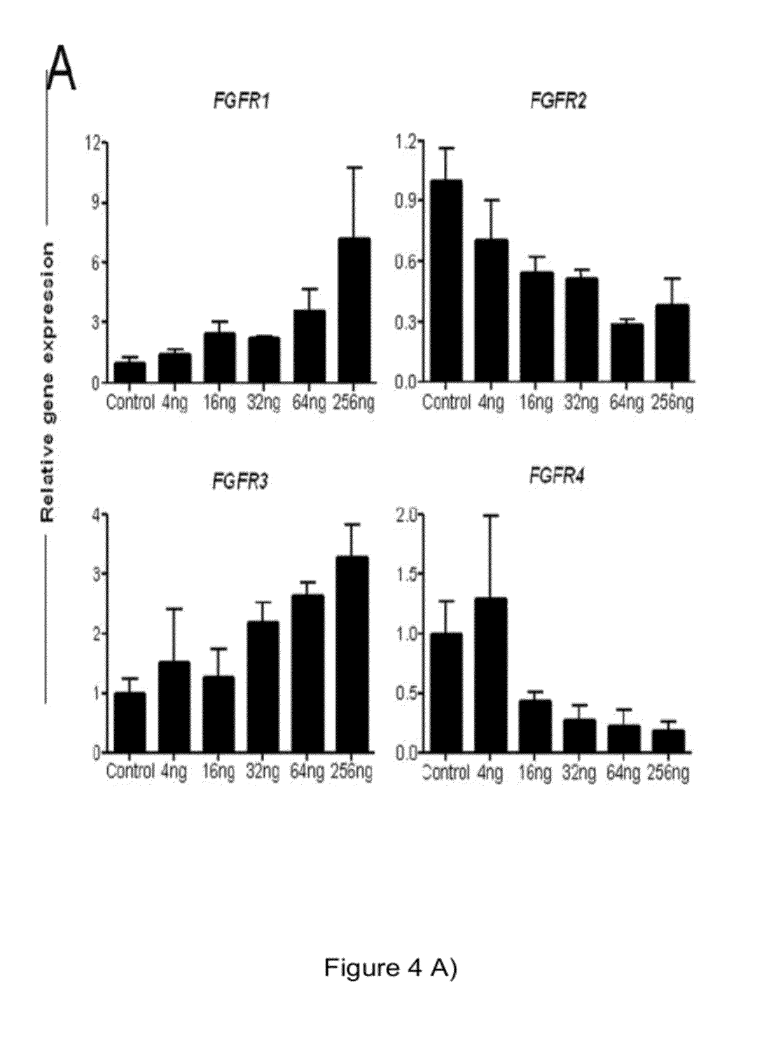

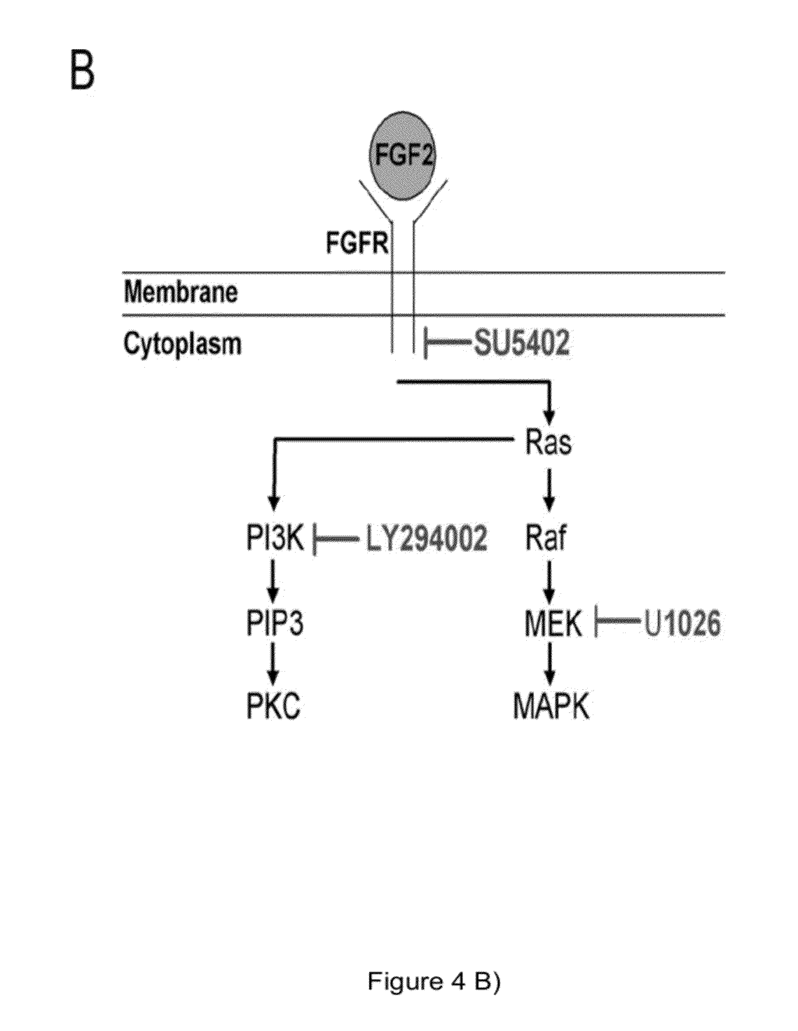

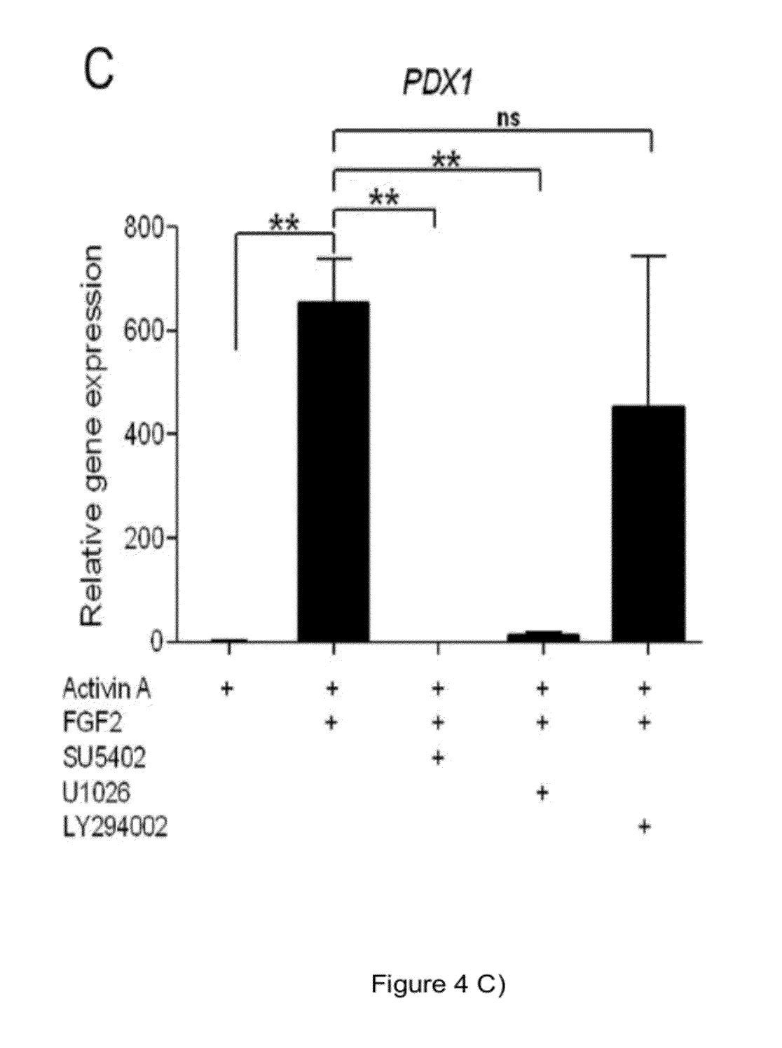

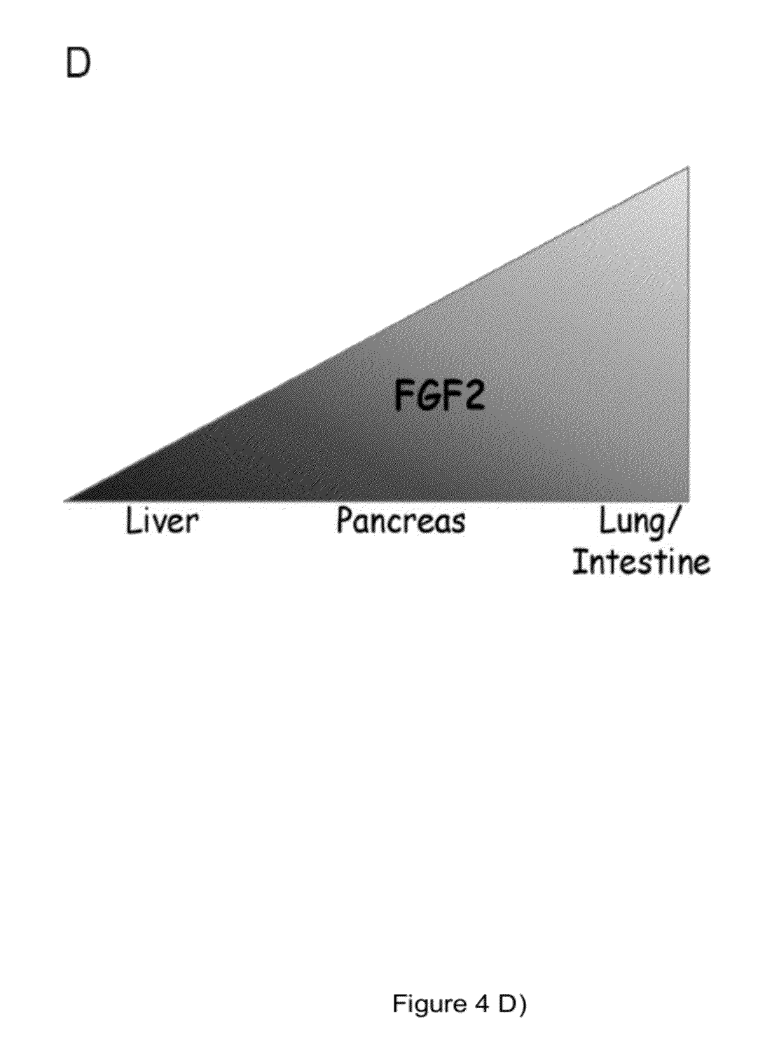

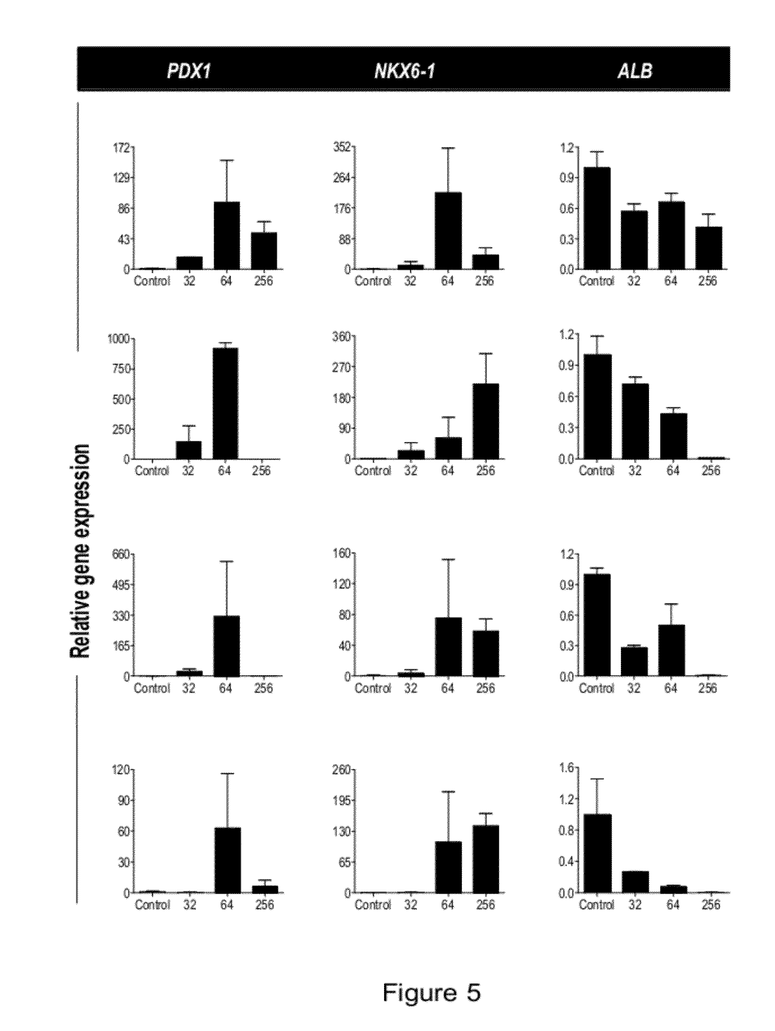

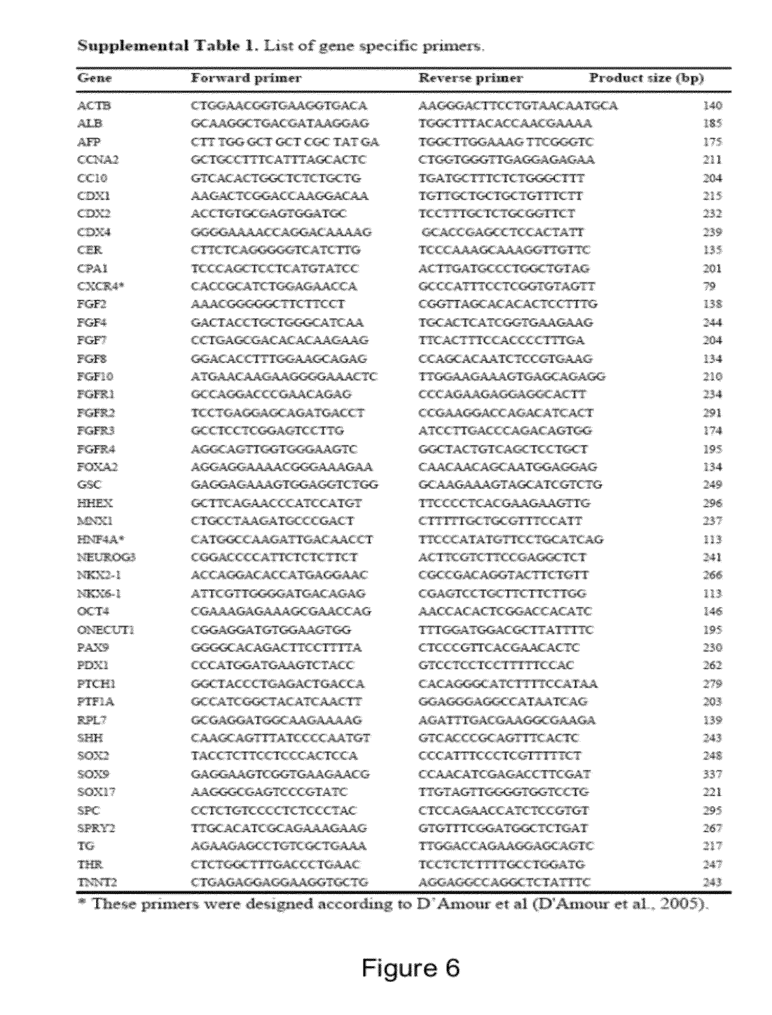

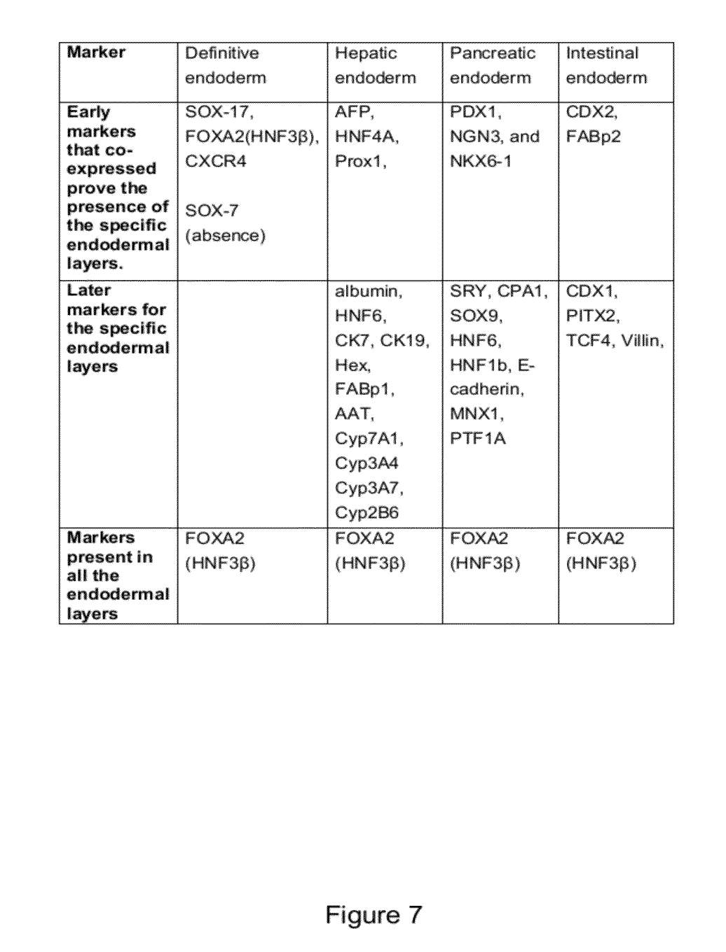

B) Quantified PDX1 immunofluorescence stainings of hPS cells treated with different FGF2 concentrations. PDX1+ cells are absent in cultures treated only with Activin A or 4 ng/ml FGF2, while in the cultures treated with 32, 64 and 256 ng/ml FGF2, PDX1+ cells are always present. The highest percentage of PDX1+ cells was observed at 64 ng/ml. This was assessed both by microscopy and the use of the Imaris Imaging software as quantified by bars in FIG. 2B). The data is presented as the mean+SEM (n =7-10). Following P-values were attained: control vs. 32 ng/ml (P<0.01), control vs. 64 ng/ml (P<0.001), control vs. 256 ng/ml (P<0.001), 32 ng/ml vs. 64 ng/ml (P<0.001), 32 ng/ml vs. 256 ng/ml (P<0.01) and 64 ng/ml vs. 256 ng/ml (P<0.01). P<0.05 was considered to be significant.

FIG. 3. RNA analysis of lung and intestinal specific markers. The anterior foregut specific marker SOX2 was significantly upregulated at 256 ng/ml, while lung associated markers such as NKX2-1, SHH, PTCH1, SPRY2, and FGF10 all had a peak expression at 256 ng/ml.

The small intestinal marker CDX1 remained unaffected, while CDX2, another marker of small intestine, and MNX1 were, however, both upregulated at 256 ng/ml. Samples were taken for real-time PCR analysis at day eleven. The data is shown as mean expression +/−SEM (n=4).

The graphs represent the fold increase in comparison to that detected in the control samples at day eleven. The control sample was arbitrarily set to a value of one.

FIG. 4. A) FGF receptor expression at day eleven. FGFR1 and FGFR3 expression were upregulated with higher FGF2 concentration, at the same time FGFR2 and 4 were downregulated. All samples for real-time PCR analysis were taken at day eleven. The data is shown as mean expression +/−SEM, n=3-4. The graphs represent the fold increase in comparison to that detected in the control samples at day eleven. The control sample was arbitrarily set to a value of one.

B) Schematic view of the intracellular signaling pathways, activated by FGF2, and their corresponding inhibitors, shown in red. C) Inhibition of FGF signaling diminished PDX1 expression in vitro. Antagonizing FGF signaling with SU5402 (10 μM) or the MAPK inhibitor, U1026 (10 μM) resulted in significantly reduced PDX1 expression while treatment with the PI3K inhibitor LY294002, (12.5 μM) had no significant effect on the PDX1 expression. The data is shown as mean expression +/−SEM, n=4-6. The graphs represent the fold increase in comparison to that detected in the control samples at day eleven. The control sample was arbitrarily set to a value of one.

D) Schematic drawing showing the different FGF2 thresholds needed to give rise to liver, pancreas and lungs. Low FGF2 concentrations promote differentiation towards hepatocyte-like cells (marked by ALB expression), while moderate FGF2 levels differentiate the hPS cell-derived foregut endoderm into pancreas (marked by PDX1 expression), whereas high concentrations promote differentiation towards pulmonary and intestinal cells (marked by NKX2-1 and CDX2 expression).

FIG. 5. RNA expression analysis of PDX, NKX6-1, and Alb in four independent experiments using four different cell lines. In all experiments, PDX1 expression was upregulated in the FGF2 treated samples compared to the control (only AA treated) except at 256 ng/ml where it was either downregulated or abolished. Furthermore, peak expression of PDX1 was always at 64 ng/ml. NKX6-1 expression was also upregulated with higher FGF2 concentration, however, it was not downregulated at 256 ng/ml in SA121 tryp, HUES-4, and HUES15, which was the case in HUES-3 and SA181tryp at day eleven. Alb expression was consistently downregulated with higher FGF2 concentrations. Upper panel shows data from cell line SA181 tryp, SA121tryp, and lower panel: HUES-4 and HUES15. Samples were taken for real-time PCR analysis at day eleven. The data is shown as mean expression +/−SEM (n=2-3). The graphs represent the fold increase in comparison to that detected in the control samples at day eleven. The control sample was arbitrarily set to a value of one.

FIG. 6. List of gene-specific primers used for PCR and gene-expression analysis.

FIG. 7. Cellular markers characteristic for definitive endoderm, hepatic endoderm, pancreatic endoderm and intestinal endoderm.

Abbreviations

AA; Activin A

Albumin (ALB)

alpha-fetoprotein (AFP)

Caudal type homeobox 2 (CDX2)

Chemokine (C-X-C motif) receptor 4 (CXCR4)

Definitive endoderm (DE)

FBS; fetal bovine serum

FGF2; Fibroblast growth factor 2

Fibroblast growth factor (FGF)

Forkhead box A2 (FOXA2)

Hematopoietically expressed homeobox (HHEX)

Hepatocyte nuclear factor 4, alpha (HNF4A)

hBS cells; human blastocyst-derived stem cells

hPS cells; human pluripotent stem cells

KO-SR; knockout serum replacement.

Pancreatic and duodenal homeobox 1 (PDX1)

Motor neuron and pancreas homeobox 1 (MNX1)

NK2 homeobox 1 (NKX2-1)

NK6 homeobox 1 (NKX6-1)

Sonic hedgehog homolog (Drosophila) (SHH),

SRY (sex determining region Y)-box 9 (SOX9)

SRY (sex determining region Y)-box 17 (SOX17)

### DEFINITIONS

As used herein, “human pluripotent stem cells” (hPS) refers to cells that may be derived from any source and that are capable, under appropriate conditions, of producing human progeny of different cell types that are derivatives of all of the 3 germinal layers (endoderm, mesoderm, and ectoderm). hPS cells may have the ability to form a teratoma in 8-12 week old SCID mice and/or the ability to form identifiable cells of all three germ layers in tissue culture. Included in the definition of human pluripotent stem cells are embryonic cells of various types including human blastocyst derived stem (hBS) cells in literature often denoted as human embryonic stem (hES) cells, (see, e.g., Thomson et al. (1998), Heins et. al. (2004), as well as induced pluripotent stem cells (see, e.g. Yu et al., (2007) Science 318:5858); Takahashi et al., (2007) Cell 131(5):861). The various methods and other embodiments described herein may require or utilise hPS cells from a variety of sources. For example, hPS cells suitable for use may be obtained from developing embryos. Additionally or alternatively, suitable hPS cells may be obtained from established cell lines and/or human induced pluripotent stem (hiPS) cells.

As used herein “hiPS cells” refers to human induced pluripotent stem cells.

As used herein, the term “blastocyst-derived stem cell” is denoted BS cell, and the human form is termed “hBS cells”. In literature the cells are often referred to as embryonic stem cells, and more specifically human embryonic stem cells (hESC). The pluripotent stem cells used in the present invention can thus be embryonic stem cells prepared from blastocysts, as described in e.g. WO 03/055992 and WO 2007/042225, or be commercially available hBS cells or cell lines. However, it is further envisaged that any human pluripotent stem cell can be used in the present invention, including differentiated adult cells which are reprogrammed to pluripotent cells by e.g. the treating adult cells with certain transcription factors, such as OCT4, SOX2, NANOG, and LIN28 as disclosed in Yu, et al., 2007, Takahashi et al. 2007 and Yu et al 2009.

As used herein feeder cells are intended to mean supporting cell types used alone or in combination. The cell type may further be of human or other species origin. The tissue from which the feeder cells may be derived include embryonic, fetal, neonatal, juvenile or adult tissue, and it further includes tissue derived from skin, including foreskin, umbilical chord, muscle, lung, epithelium, placenta, fallopian tube, glandula, stroma or breast. The feeder cells may be derived from cell types pertaining to the group consisting of human fibroblasts, fibrocytes, myocytes, keratinocytes, endothelial cells and epithelial cells. Examples of specific cell types that may be used for deriving feeder cells include embryonic fibroblasts, extraembryonic endodermal cells, extraembryonic mesoderm cells, fetal fibroblasts and/or fibrocytes, fetal muscle cells, fetal skin cells, fetal lung cells, fetal endothelial cells, fetal epithelial cells, umbilical chord mesenchymal cells, placental fibroblasts and/or fibrocytes, placental endothelial cells,

As used herein, the term “mEF cells” is intended to mean mouse embryonic fibroblasts.

As used herein, the term “small molecules” is intended to mean compounds that activate a preferred signalling pathway.

### EXAMPLES

### Example 1

In Vitro Culture of Human ES Cells

Undifferentiated hPSs (trypsin adapted SA181 and SA121 (Cellartis, Gothenburg, www.cellartis.com), HUES-3, HUES-4, and HUES-15 obtained from D. A. Melton, Howard Hughes Medical Institute (Harvard University, Cambridge, Mass.)(Cowan et al., 2004)) were propagated as previously described (Cowan et al., 2004; Heins et al., 2004), protocols are also available at http://mcb.harvard.edu/melton/hues/. Briefly, cells were maintained on mitotically inactivated mouse embryonic fibroblasts (MEFs) (Department of Experimental Biomedicine/TCF from Sahlgrenska Academy at the University of Gothenburg, Sweden) in hBS medium containing KO-DMEM, 10% knockout serum replacement, 10 ng/ml bFGF, 1% non-essential amino acids, 1% Glutamax, 1% Penicillin-streptomycin, beta-Mercaptoethanol (all reagents from GIBCO, Invitrogen) and 10% plasmanate (Talecris Biotherapeutics Inc). Cells were passaged with 0.05% trypsin/EDTA (GIBCO, Invitrogen) and re-plated at a split-ratio between 1:3 and 1:6. Cell lines were karyotyped by standard G-banding by the Institute of Clinical Genetics, University of Linkoping, Sweden. For each analysis, 15-20 metaphases were evaluated. SA121, HUES-4, and HUES-15 were karyotypically normal, whereas HUES-3 (subclone 52) had gained an extra chromosome 17 (82%) and SA181 had gained an extra chromosome 12 (45%).

### Example 2

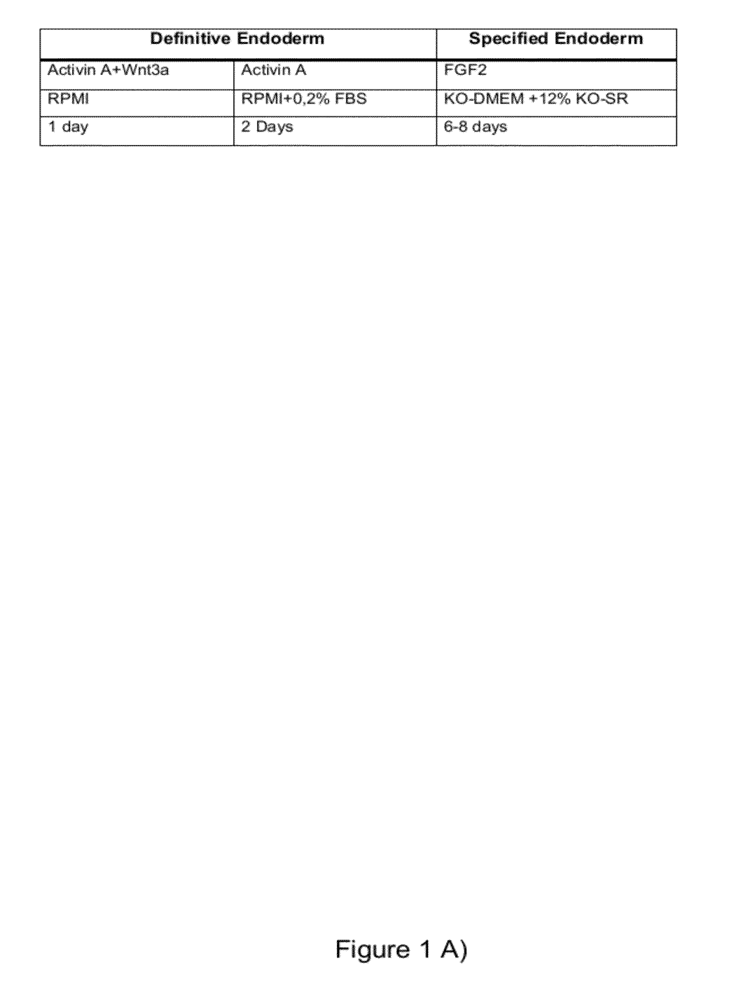

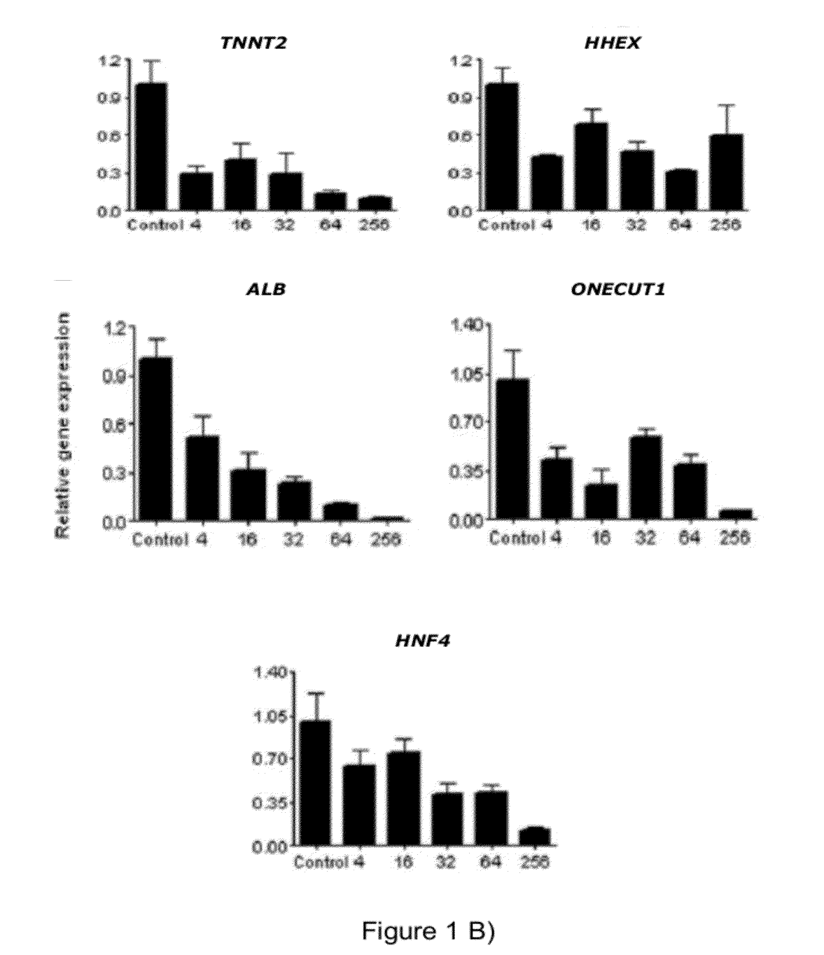

Differentiation of hPS Cells into Definitive Endodermal Cells and Specific Endoderm Cells According to FIG. 1

hPS cells were seeded at a density of 12,000-24,000 cells/cm² and cultured until confluence. hPS cells were then differentiated into definitive endoderm as described previously (D'Amour et al., 2005). Briefly, cells were washed in PBS and treated with 100 ng/ml Activin A (R&D systems) and 25 ng/ml Wingless-type MMTV integration site family, member 3A (Wnt3a) in RPM! 1640 (GIBCO, Invitrogen) for three days in low serum (0-0.2% FBS).

At day three, cells were washed with PBS and human FGF2 (Invitrogen) was added at different concentrations (0-256 ng/ml according to specifications in the results) in a KO-DMEM based medium containing 1% Penicillin-streptomycin, 1% Glutamax, 1% non-essential amino acids, 0.1mM beta-Mercaptoethanol and 12% knockout serum replacement (all reagents from Invitrogen). Medium was changed every day. Control cultures without FGF2 were grown in parallel and cell morphology was monitored daily. At each time point, two to four biological replicates were taken for each independent experiment. More specifically, each well was divided into 4-5 equal pieces depending on the number of time points that were analyzed.

### Example 3

Characterisation of Specific Endodermal Cells

FGF Inhibition Assays

FGF receptor inhibition assays were performed by adding SU5402 (Calbiochem; 10 M), LY294002 (Cell Signalling technology; 12.5 μM) and U1026 (Cell Signalling technology; 10 μM) to the medium following DE induction at day three. Control cultures were treated with equal volume of the diluent DMSO. Fresh medium supplemented with appropriate inhibitor was added daily. Two to three samples were taken from separate wells at different time points (day 9-12) for mRNA analysis for each independent experiment.

RNA Extraction, Reverse Transcription and Real-Time PCR

Total RNA was extracted with GenElute Mammalian total RNA kit (Sigma-Aldrich). Total RNA concentrations were measured with the NanoDrop ND-1000 spectrophotometer (Nanodrop Technologies). Reverse transcription was performed with SuperScript III, according to the manufacturer's instructions, using 2.5 μM random hexamer and 2.5 μM oligo(dT) (Invitrogen). Real-time PCR measurements were performed on an ABI PRISM 7900HT Sequence Detector System (Applied Biosystems). 20 μl reactions containing 10 μl SuperMix-UDG w/ROX, 400 nM of each primer, 0.125× SYBR Green I (all reagents from Invitrogen) were used. Primer sequences are available as supplementary data (FIG. 6). Formation of expected PCR products was confirmed by agarose gel electrophoresis and melting curve analysis. Gene expression data was normalized against ACTB or RPL7 expression. As an extra normalization control, data was also normalized against total RNA concentrations, which resulted in similar data. Real-time PCR data analysis was performed as described (Bustin, 2000; Stahlberg et al., 2005).

Immunohistochemical Analysis of hPS Cells

hPS cells were fixed in 4% paraformaldehyde for 15 minutes at room temperature and washed three times in PBS-T (0.1% Triton X-100 in PBS). Fixed cells were permeabilized with 0.5% Triton X-100 in PBS for 15 minutes and blocked in PBS-T supplemented with 5% normal donkey serum (Jackson lmmunoresearch) for 1 h at room temperature before they were incubated overnight at 4° C. with the following primary antibodies and dilutions: goat polyclonal antibody (pAb) anti-FOXA-2 (kind gift from Palle Serup; Santa Cruz Biotechnology; 1:200), Guinea Pig pAb anti-PDX-1 (Chris Wright; BetaCellBiologyConsortium; 1:1500), Goat anti-PDX-1 (Chris Wright; BetaCellBiologyConsortium; 1:1500), rabbit pAb anti-NKX6.1 (BetaCellBiologyConsortium; 20 1:4000), mouse anti-CDX-2 (kind gift from Jonathan Draper; Biogenex; 1:500), rabbit pAb anti-SOX-9 (Chemicon; 1:500), rabbit anti-HNF-6 (Santa Cruz Biotechnology; 1:400), mouse mAb-anti PH-3 (Cell Signaling technology; 1:50), rabbit pAb-anti MKi67 (Novocastra; 1:200), rabbit anti-S0X2 (kind gift from Palle Serup; Chemicon; 1:250), goat anti-albumin (Bethyl laboratories; 1:300). After overnight incubation cells were washed three times for 5 minutes in PBS; and incubated with corresponding fluorescent secondary antibodies (Alexa 488, Cy3 and 647; Jackson lmmunoresearch and Invitrogen; diluted according to the manufacturer's instructions) for 60 min in PBS-T supplemented with 5% serum at room temperature. Cell nuclei were visualized by 4′-6′diamidino-2-phenylindole (DAPI) (Sigma-Aldrich; 1:1000) incubation for 4 minutes. Immunofluorescence stainings were detected and analyzed by epifluorescence microscopy (Zeiss Axioplan 2).

Data Analysis

The percentage of PDX1 positive cells was calculated using the Imaris Imaging software (Bitplane). Ten randomly selected fields were chosen for each parameter. Using DAPI staining the software estimated the total area of cells. The area of the PDX1 positive cells was calculated in the same manner. Finally, the percentage of PDX1 positive cells was calculated by dividing the area of PDX1 positive cells by the DAPI positive area. Raw data from realtime PCR measurements was exported from SDS 2.2.1 and analyzed by Microsoft Excel graph pad. All data were statistically analyzed by multivariate comparison (one-way ANOVA) with Bonferroni correction. All values are depicted as mean±standard error of the mean (SEM) and considered significant if p<0.05.

### Example 4

Low Doses of FGF2 Promote a Hepatic Cell Fate while Intermediate FGF2 Concentrations Direct Differentiation of hPS Cells Towards a Pancreatic Cell Fate

For the present invention it was examined whether Activin A/Wnt3a-treated hPS cells were capable of giving rise to both anterior and posterior foregut endoderm, from where the ventral and dorsal pancreas originates, respectively. Indeed, by assessing the expression of characteristic foregut/midgut markers, we show that Activin A/Wnt3a-treated hPS cells spontaneously differentiate into foregut and midgut endoderm (FIG. 1B). Furthermore, of the foregut-derived organs, liver progenitors predominated (FIG. 1B,2A). Altogether, these findings suggest that anterior foregut endoderm spontaneously differentiates into liver but that neither anterior nor posterior foregut endoderm spontaneously become specified into the ventral and dorsal pancreatic endoderm, respectively. To test whether FGF2 is capable of directing differentiation of foregut endoderm into a pancreatic fate, the ability of different FGF2 concentrations (0, 4, 16, 32, 64 and 256 ng/ml) to induce PDX1-expression was assessed. The concentrations were partially based on mouse explant studies (Deutsch et al., 2001). The differentiation protocol (FIG. 1A) was applied on five different cell lines, HUES-3: subclone 52, HUES-4, HUES-15, and the trypsin-adapted SA181 and SA121 to avoid cell line specific optimization. Cells treated with FGF2 concentrations (16-256 ng/ml) grew denser and contained more clusters. Hepatocyte-like cells were seen in the hPS cell cultures treated with low doses of FGF2 (4 ng/ml). mRNA analysis and immunofluorescence stainings revealed a dose-dependent expression of the hepatic markers albumin (ALB), one cut homeobox 1 (ONECUT1 previously known as HNF6), hepatocyte nuclear factor 4 alpha (HNF4A), whereas HHEX expression was only moderately reduced in a non-dose-dependent manner (at least within the range of tested FGF2 concentrations). Increased FGF2 concentration downregulated the expression of ALB, ONECUT1, and HNF4A . This was also confirmed at the protein level by ALB stainings, where abundant ALB+cells were seen at 0 and 4 ng/ml FGF2 and none at 256 ng/ml FGF2 (FIG. 1B).

Multiple transcription factors are known to be involved in pancreas specification. However, most of these factors are also expressed in other organs. Hence, a combination of markers was chosen to determine pancreatic fate of differentiated cells: PDX1, SRY (sex determining region Y)-box 9 (50X9), NK6 homeobox 1 (NKX6-1), the bHLH transcription factors Neurogenin-3 (NGN3), FOXA2, and Carboxypeptidase A1 (CPA1) expression was also monitored. Expression of posterior foregut associated markers was detected in all samples, and expression of several pancreatic endodermal markers, including PDX1, NKX6-1, SOX9, and NGN3, was upregulated in a FGF2 dose-dependent manner. Low levels of NKX6-1 could in the majority of the experiments be detected already at day nine but expression become more evident from day eleven onwards. CPA1 and FOXA2 were expressed in all samples but not influenced by FGF2 treatment (FIG. 2A, Supp. FIG. 1).

Expression analysis of the pancreas specific transcription factor 1a (PTF1A), a member of the basic helix-loop-helix (bHLH) transcription factor family, which is expressed in the early pancreatic endoderm was expressed at low mRNA levels (data not shown).

As all pancreatic tissue is derived from a Pdx1 positive population and to confirm the mRNA data, PDX1 stainings were performed. We detected PDX1+ cells exclusively in samples treated with 32-256 ng/ml FGF2 (FIG. 2B). The number of PDX1+ cells was significantly higher for FGF2-treated cells (32-256 ng/ml) compared to control cells that were not treated with FGF2. The highest number of PDX1+ cells (15-20%) was obtained in cultures treated with 64 ng/ml FGF2 (FIG. 2B). Although the effect of the highest FGF2 concentration varied between cell lines, the tendency was the same; PDX1 expression was either decreased or abolished at 256 ng/ml (Supp. FIG. 1).

As Pdx1 is also expressed in the posterior stomach, duodenum, and CNS (only mRNA transcript), expression of additional pancreatic markers was used to verify differentiation towards a pancreatic fate. All PDX1+ cells co-expressed FOXA2, ONECUT1, and SOX9. Although the vast majority of the PDX1+ cells did not coexpress the midgut/hindgut marker CDX2, a few double positive cells were detected. PDX1 and NKX6-1 are co-expressed in mouse and human pancreatic epithelium but not in the duodenum and stomach (Nelson et al., 2007). Pancreatic progenitors co-expressing PDX1 and NKX6-1 were only found in samples treated with 32 ng/ml and 64 ng/ml FGF2 respectively (FIG. 2A). However, the number of NKX6-1+ cells was relatively small in comparison to the PDX1+ population. Robust induction of PDX1 expression at 32-256 ng/ml FGF2 was reproduced in multiple experiments using five different hPS cell lines (Supp. FIG. 1). Thus, increasing FGF2 concentration favored a pancreatic cell fate at the expense of a hepatic cell fate (FIG. 2A and Supp. FIG. 1). Immunofluorescence detection of the proliferation marker phospho-histone-H3 (PH3) demonstrated that only few PDX1+ cells replicated, suggesting that the appearance of PDX1+ cells is the result of differentiation rather than proliferation of pre-existing PDX1+ cells.

### Example 5

High Doses of FGF2 Direct Differentiation of hPS Cells into Anterior Foregut and Small Intestinal Cells

As the expression of the hepatocyte markers ALB, HNF4A, and ONECUT1 decreased with increasing FGF2 concentration (FIG. 1B), the expression level of the anterior foregut associated marker SRY (sex determining region Y)-box 2 (SOX-2) increased, with the highest level seen at 256 ng/ml (FIG. 2A). Consistently, Sox-2 expression was confined to anterior foregut-derivatives, such as esophagus, lung and stomach, in the E13.5 mouse embryo (Supp. FIG. 2). Since lung and thyroid arise from the same region of anterior foregut endoderm, the expression pattern of markers associated with these organs was assessed by mRNA analysis. While the thyroid-specific marker thyroglobulin (TG) was downregulated with increasing FGF2 concentrations (data not shown), the earliest marker of lung and thyroid specification NKX2-1 (Serls et al., 2005) was upregulated at 256 ng/ml, suggesting differentiation to pulmonary cell types. Additional markers associated with, but not restricted to, the induction of a pulmonary fate, such as fibroblast growth factor 10 (FGF10), sprouty homolog 2 (Drosophila) (SPRY2), sonic hedgehog homolog (Drosophila) (SHH) and the SHH receptor patched homolog 1 (Drosophila) (PTCH1),were also upregulated (FIG. 3).

The pulmonary surfactant protein C (SP-C), produced by the alveolar Type II epithelial cells and Clara cell 10 kDa protein (CC10) could not be detected in the mRNA samples, suggesting that the NKX2-1+ cells represent early lung progenitor cells.

Expression of the midgut/hindgut markers CDX2 and MNX1 significantly increased at the highest FGF2 concentration (256 ng/ml), suggesting that high concentration of FGF2 also induced formation of intestinal cell types. CDX1 expression remained unchanged whereas the large intestine marker CDX4 was not detected at any concentration. CDX2 expression was confirmed at protein level and the highest number of CDX2+ cells was obtained at 256 ng/ml. Importantly, CDX2+ cells co-expressed FOXA2, excluding formation of trophectoderm. To determine if the increased number of CDX2+ cells was a result of proliferation or re-specification of midgut endoderm, double stainings with the proliferation marker MKI67 were carried out. The majority of CDX2+ cells were negative for the MKI67 antigen, implicating re-specification rather than proliferation.

Although many PDX1+ cells were still expressed at 256 ng/ml FGF2, none of them expressed NKX6-1, suggesting that increasing the FGF2 concentration from 64 to 256 ng/ml blocked formation of pancreatic endoderm (FIG. 5). Furthermore, while the majority of the PDX1+ cells were CDX2 negative, more PDX1+/CDX2+ cells were seen at 256 ng/ml compared to 64 ng/ml. Based on PDX1/CDX2 double stainings of E18.5 mouse embryos, we conclude that PDX1+/CDX2+ cells represent duodenal cell types. Additionally, we could confirm that neither PDX1 nor the CDX2 positive cells co-expressed SOX2 in the differentiated hPS cells and in the E18.5 mouse embryos. In summary, these data suggest a dose-dependent induction of the hepatic, pancreatic, pulmonary, and intestinal markers in response to exogenous FGF2 (FIG. 4D).

### EXAMPLE 6

ERK1/2 Mitogen-Activated Protein Kinase Signalling is Required for PDX1 Induction

FGFs activate through their corresponding FGFRs several signal transduction pathways, including phosphatidylinositol-3 kinase (PI3K) and ERK1/2 mitogen-activated protein kinases (MAPKs) (FIG. 4B). FGFR mRNA expression was detected in all samples. Furthermore, a tendency towards elevated levels of FGFR1 and 3 and decreased levels of FGFR2 and 4 was seen with increasing FGF2 concentration (FIG. 4A). To determine whether FGFR-mediated signalling is required for differentiation towards pancreatic endoderm, the effect of the FGFR tyrosine kinase inhibitor SU5402, MAPK inhibitor U1026, and PI3K inhibitor LY294002, was investigated (FIG. 4C). Treatment with SU5402 significantly decreased the number of PDX1 positive cells suggesting that FGF2 (64 ng/ml) mediates induction of PDX1+ cells through FGFRs. In addition, treatment with FGF2 in the presence of U1026 diminished PDX1 expression, indicating that activation of the MAPK pathway by FGFR signalling is necessary for induction of PDX1. In contrast, when cells were treated with FGF2 in the presence of LY294002, PDX1 expression remained unchanged, suggesting that an active PI3K pathway is not required for induction of PDX1. These results demonstrate that FGF2 induced PDX1 expression in the hPS cells relies on the specific activation of the MAPK pathway downstream of FGFR signalling.

### REFERENCES

Ang, S. L., Wierda, A., Wong, D., Stevens, K. A., Cascio, S., Rossant, J. and Zaret, K. S. (1993). The formation and maintenance of the definitive endoderm lineage in the mouse: involvement of HNF3/forkhead proteins. Development 119, 1301-15.

Bustin, S. A. (2000). Absolute quantification of mRNA using real-time reverse transcription polymerase chain reaction assays. J Mol Endocrinol 25, 169-93.

Cowan, C. A., Klimanskaya, I., McMahon, J., Atienza, J., Witmyer, J., Zucker, J. P., Wang, S., Morton, C. C., McMahon, A. P., Powers, D. et al. (2004). Derivation of embryonic stem-cell lines from human blastocysts. N Engl J Med 350, 1353-6.

D'Amour, K. A., Agulnick, A. D., Eliazer, S., Kelly, O. G., Kroon, E. and Baetge, E. E. (2005). Efficient differentiation of human embryonic stem cells to definitive endoderm. Nat Biotechnol 23, 1534-41.

Serls, A. E., Doherty, S., Parvatiyar, P., Wells, J. M. and Deutsch, G. H. (2005). Different thresholds of fibroblast growth factors pattern the ventral foregut into liver and lung. Development 132, 35-47.

Stahlberg, A., Zoric, N., Aman, P. and Kubista, M. (2005). Quantitative real-time PCR for cancer detection: the lymphoma case. Expert Rev Mol Diagn 5, 221-30.

Takahashi K, Tanabe K, Ohnuki M, Narita M, Ichisaka T, Tomoda K and Yamanaka S, Induction of Pluripotent Stem Cells from Adult Human Fibroblasts by Defined Factors, Cell 131, 861-872, Nov. 30, 2007

Yu J, Vodyanik M A, Smuga-Otto K, Antosiewicz-Bourget J, Frane J L, Tian S, Nie J, Jonsdottir G A, Ruotti V, Stewart R, Slukvin II, Thomson J A, Induced Pluripotent Stem Cell Lines Derived from Human Somatic Cells, Science vol 318 21 Dec. 2007

Yu J, Hu K, Smuga-Otto K, Tian S, Stewart R, Slukvin II, Thomson J A, Human Induced Pluripotent Stem Cells Free of Vector and Transgene Sequences, Science vol 324 8 May 2009

1-33. (canceled)

34. A method to control differentiation of definitive endodermal cells derived from human pluripotent stem (hPS) cells comprising:
providing a concentration of fibroblast growth factor 2 (FGF2) to control differentiation of definitive endodermal cells derived from hPS cells to specific endoderm cells.

35. The method of claim 34, wherein the hPS cells are human blastocyst derived stem (hBS) cells.

36. The method of claim 34, wherein the concentration of FGF2 in a culture medium is less than or equal to 500 ng/ml.

37. The method of claim 34, wherein the concentration of FGF2 in a culture medium ranges from about 16 ng/ml to about 150 ng/ml, and wherein the specific endoderm cells are pancreatic endoderm cells.

38. The method of claim 34, wherein the concentration of FGF2 in a culture is 64 ng/ml, and wherein the specific endoderm cells are pancreatic endoderm cells.

39. The method of claim 37, wherein the pancreatic endoderm cells express PDX1, and one or more of the following markers NGN3, CPA1, SOX9, HNF6, HNF1b, Ecadherin, MNX1, PTF1A and NKX6-1.

40. The method of claim 39, wherein the pancreatic endoderm cells express PDX1 and NKX6-1.

41. The method of claim 37, wherein the pancreatic endoderm cells express the following markers: SOX9, ONECUT1, and FOXA2.

42. The method of claim 39, wherein the pancreatic endoderm cells express the following markers: SOX9, ONECUT1, and FOXA2.

43. The method of claim 37, wherein the pancreatic endoderm cells express at least one pancreatic hormone selected from the group consisting of insulin, glucagon, somatostatin, pancreatic polypeptide, and ghrelin.

44. The method of claim 34, wherein the differentiation comprises incubation of definitive endoderm cells in a culture medium containing FGF2 in a concentration that is suitable for differentiation in the desired endodermal fate selected from the group consisting of hepatic endoderm cells, pancreatic endoderm cells, intestinal endoderm cells, and lung endoderm cells.

45. A method for the preparation of pancreatic endodermal cells, the method comprising incubating definitive endodermal cells in a culture medium comprising from about 16 ng/ml to about 150 ng/ml FGF2 for about 2 to about 20 days.

46. The method of claim 45, wherein incubating definitive endodermal cells in a culture mediumis occurs for about 6 to about 8 days.

47. Pancreatic endodermal cells obtainable by the method of claim 45.

48. The pancreatic endodermal cells of claim 47, wherein the pancreatic endoderm cells express PDX1, and one or more of the following markers NGN3, CPA1, SOX9, HNF6, HNF1b, Ecadherin, MNX1, PTF1A and NKX6-1.

49. The pancreatic endodermal cells of claim 48, wherein the pancreatic endoderm cells express PDX1 and NKX6-1.

50. The pancreatic endodermal cells of claim 48, wherein the pancreatic endoderm cells express the following markers: SOX9, ONECUT1, and FOXA2.

51. The pancreatic endodermal cells of claim 47, wherein the pancreatic endoderm cells express the following markers: SOX9, ONECUT1, and FOXA2.

52. The method of claim 37, wherein the pancreatic endoderm cells comprises progenitor cells that express at least one marker for proliferation selected from the group consisting of MKI67, PH3, Brdu.
